(19) United States
(12) Patent Application Publication
(10) Pub. No.: US 2006/0148891 A1
(43) Pub. Date: Jul. 6, 2006

(54) Formulations of thiomolybdate or thiotungstate compounds and uses thereof

(76) Inventors: Robert J. Ternansky, San Diego, CA (US); Brian W. Sullivan, Escondido, CA (US); Andrew X. Chen, San Diego, CA (US)

Correspondence Address: Richard F. Trecartin;Dorsey & Whitney LLP, Intellectual Property Department, 555 California Street, 10th Floor, San Francisco, CA, 94104-1513, US

(21) Appl. No.: 11/256,540

(22) Filed: Oct. 21, 2005

Related U.S. Application Data
(63) Continuation of application No. 11/064,740, filed on Feb. 23, 2005, now abandoned.
(60) Provisional application No. 60/547,357, filed on Feb. 23, 2004.

(51) Int. Cl. A61K 31/28

(52) U.S. Cl. 514/492

(57) ABSTRACT

Formulations of thiomolybdate or thiotungstate derivatives, methods of making formulations of thiomolybdate or thiotungstate derivatives, solid dosage forms of thiomolybdate or thiotungstate analogues, methods of making solid dosage forms of thiomolybdate or thiotungstate analogues and methods of using solid dosage forms of thiomolybdate or thiotungstate derivatives to treat or prevent diseases associated with aberrant vascularization or aberrant angiogenesis, copper metabolism disorders neurodegenerative disorders and obesity are disclosed herein.

This application claims the benefit under 35 U.S.C. § 119(e) from U.S. Provisional Application Ser. No. 60/547,357, filed Feb. 23, 2004 and this application is a continuation of U.S. application Ser. No. 11/064,740, filed Feb. 23, 2005 which is herein incorporated by reference in its entirety.

### 1. FIELD

The present invention relates generally to formulations of thiomolybdate or thiotungstate derivatives, methods of making formulations of thiomolybdate or thiotungstate derivatives, solid dosage forms of thiomolybdate or thiotungstate analogues, methods of making solid dosage forms of thiomolybdate or thiotungstate analogues and methods of using solid dosage forms of thiomolybdate or thiotungstate derivatives to treat or prevent diseases associated with aberrant vascularization or aberrant angiogenesis, copper metabolism disorders neurodegenerative disorders and obesity.

### 2. BACKGROUND

Most forms of cancer are derived from solid tumors (Shockley et al., Ann. N.Y. Acad. Sci. 1991, 617: 367-382), which have proven resistant in the clinic to therapies such as the use of monoclonal antibodies and immunotoxins. Anti-angiogenic therapy for the treatment of cancer was developed from the recognition that solid tumors require angiogenesis (i.e., new blood vessel formation) for sustained growth (Folkman, Ann. Surg. 1972, 175: 409-416; Folkman, Mol. Med. 1995, 1(2): 120-122; Folkman, Breast Cancer Res. Treat. 1995, 36(2): 109-118; Hanahan et al., Cell 1996, 86(3): 353-364). Efficacy of anti-angiogenic therapy in animal models has been demonstrated (Millauer et al., Cancer Res. 1996, 56:1615-1620; Borgstrom et al., Prostrate 1998, 35: 1-10; Benjamin et al., J. Clin. Invest. 1999, 103: 159-165; Merajver et al., Proceedings of Special AACR Conference on Angiogenesis and Cancer 1998, Abstract #B-11, January 22-24). In the absence of angiogenesis, internal cell layers of solid tumors are inadequately nourished. Further, angiogenesis (i.e., aberrant vascularization) has been implicated in numerous other diseases (e.g., ocular neovascular disease, macular degeneration, rheumatoid arthritis, etc.). More recently, angiogenesis inhibition has been directly correlated with adipose tissue loss and weight loss in animal models, which suggests anti-angiogenic therapy may be useful in prevention of obesity (Rupnick et al., Proc. Natl. Acad. Sci. 2002, 99:10730-10735).

Contrastingly, normal tissue does not require angiogenesis except under specialized circumstances (e.g., wound repair, proliferation of the internal lining of the uterus during the menstrual cycle, etc.). Accordingly, a requirement for angiogenesis is a significant difference between tumor cells and normal tissue. Importantly, the dependency of tumor cells on angiogenesis, when compared to normal cells, is quantitatively greater than differences in cell replication and cell death, between normal tissue and tumor tissue, which are often exploited in cancer therapy.

Angiogenesis requires copper, as has been shown by numerous studies (Parke et al., Am. J. Pathol. 1988, 137:173-178; Raju et al., Natl. Cancer Inst. 1982, 69: 1183-1188; Ziche et al., Natl. Cancer Inst. 1982, 69: 475-482; Gullino, Anticancer Res. 1986, 6(2): 153-158). Attempts at preventing angiogenesis and hence tumor growth in animal models by reducing in vivo amounts of copper have been reported in the art (Brem et al., Neurosurgery 1990, 26:391-396; Brem et al., Am. J. Pathol. 1990, 137(5): 1121-1142; Yoshida et al., Neurosurgery 1995 37(2): 287-295). These approaches incorporated both copper chelators and low copper diets.

More recently, Brewer et al., International Application No. PCT/US99/20374 have shown that the copper chelators, (e.g., tetrathiomolybdate) may be effective in treating diseases (e.g., solid tumor growth), which require angiogenesis. Even more recently, Ternansky et al., U.S. patent application Ser. No. 10/447,585 entitled “Thiomolybdate Analogues and Uses Thereof” and Ternansky et al., U.S. patent application Ser. No. 10/857,321 entitled “Thiotungstate Analogues and Uses Thereof,” have shown that thiomolybdate and thiotungstate analogues may be efficacious in treating diseases that require angiogenesis. However, many thiomolybdate and thiotungstate analogues undergo rapid degradation when exposed to moisture and/or oxygen.

Accordingly, novel formulations which stabilize thiomolybdate and thiotungstate analogues under ambient conditions are required to fully explore the potential of these compounds in preventing angiogenesis. Such formulations may meet the shelf life and storage requirements of commercially viable pharmaceutical products and accordingly may be effective in treating various diseases associated with angiogenesis such as cancer and obesity along with copper metabolism disorders neurodegenerative disorders, obesity as well as treating diseases where the NF-κB pathway is dysregulated such as inflammatory disorders.

### 3. SUMMARY

The present invention satisfies these and other needs by providing formulations of thiomolybdate or thiotungstate derivatives, methods of making formulations of thiomolybdate or thiotungstate derivatives, solid dosage forms of thiomolybdate or thiotungstate analogues, methods of making solid dosage forms of thiomolybdate or thiotungstate analogues and methods of using solid dosage forms of thiomolybdate or thiotungstate derivatives to treat or prevent diseases associated with aberrant vascularization or aberrant angiogenesis, copper metabolism disorders neurodegenerative disorders and obesity.

In a first aspect, a formulation comprising a thiomolybdate or thiotungstate compound, a pharmaceutically acceptable solvent and a matrix material is disclosed. In some embodiments, the thiomolybdate or thiotungstate compound is a compound of structural formula (I): 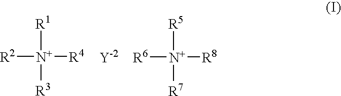
or a solvate or hydrate or N-oxide thereof wherein:
R¹, R², R³, R⁵, R⁶ and R⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl;
R⁴ and R⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl or are absent when N is part of an aromatic ring;
optionally, R¹ and R² taken together are alkyldiyl, substituted alkyldiyl, heteroalkyldiyl or substituted heteroalkyldiyl;
optionally, R⁵ and R⁶ taken together are alkyldiyl, substituted alkyldiyl, heteroalkyldiyl or substituted heteroalkyldiyl;
optionally, R¹ and R² taken together, R² and R³ taken together and R² and R⁴ taken together are alkyldiyl, substituted alkyldiyl, heteroalkyldiyl or substituted heteroalkyldiyl;
optionally, R⁵ and R⁶ taken together, R⁶ and R⁷ taken together and R⁶ and R⁸ taken together are alkyldiyl, substituted alkyldiyl, heteroalkyldiyl or substituted heteroalkyldiyl;
optionally, R³ and R⁷ taken together are alkyldiyl, substituted alkyldiyl, heteroalkyldiyl or substituted heteroalkyldiyl; and
Y⁻² is (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻² or (Mo₂S₁₃)⁻², (WS₄)⁻², (W₂S₁₂)⁻², (W₂S₉)⁻², (W₂S₇)⁻², (W₂S₈)⁻², (W₂S₁₁)⁻², (W₂S₆)⁻² or (W₂S₁₃)⁻².

In a second aspect, a solid dosage form comprising a thiomolybdate or thiotungstate compound and a matrix material is disclosed. In some embodiments, thiomolybdate or thiotungstate compound is encompassed by structural formula (I), infra. In other embodiments, the solid dosage form is a capsule form.

In a third aspect, a solid dosage form consisting essentially of a thiomolybdate or thiotungstate compound and a matrix material is provided.

In a fourth aspect, a solid dosage form made by a method where a thiomolybdate or thiotungstate compound and a matrix material and a pharmaceutically acceptable solvent are mixed together is provided. The mixture of thiomolybdate or thiotungstate compound, matrix material and pharmaceutically acceptable solvent are added to a container substantially impermeable to the solvent and the solvent is evaporated.

In a fifth aspect, a method of making a solid dosage form where a thiomolybdate or thiotungstate compound and a matrix material and a pharmaceutically acceptable solvent are mixed together is provided. The mixture of thiomolybdate or thiotungstate compound, matrix material and pharmaceutically acceptable solvent are added to a container substantially impermeable to the solvent and the solvent is evaporated.

In a sixth aspect, methods for treating or preventing diseases characterized by aberrant vascularization, copper metabolism disorders, neurodegenerative disorders and obesity are provided. The methods generally involve administering to a patient in need of such treatment or prevention a therapeutically effective amount of a solid dosage form of the invention.

### 4. DETAILED DESCRIPTION OF THE INVENTION

### 4.1 Definitions

“Compounds” refers to compounds encompassed by structural formula (I) disclosed herein and includes any specific compounds within that generic formula whose structure is disclosed herein. The compounds may be identified either by their chemical structure and/or chemical name. When the chemical structure and chemical name conflict, the chemical structure is determinative of the identity of the compound. Compounds may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds also include isotopically labeled compounds where one or more atoms have an atomic mass different from the atomic mass conventionally found in nature. Examples of isotopes that may be incorporated into the compounds include, but are not limited to, ²H, ³H, ¹³C, ¹⁴C, ¹⁵N, ¹⁸O and ¹⁷O. Compounds may exist in unsolvated forms as well as solvated forms, including hydrated forms and as N-oxides. In general, the hydrated, solvated and N-oxide forms are within the scope of the present invention. Certain compounds may exist in multiple crystalline or amorphous forms. In general, all physical forms are equivalent for the uses contemplated by the present invention and are intended to be within the scope of the present invention. Further, it should be understood, when partial structures of compounds are illustrated, that brackets indicate the point of attachment of the partial structure to the rest of the molecule.

“Alkyl” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group is from 1 to 20 carbon atoms. In other embodiments, an alkyl group is from 1 to 10 carbon atoms. In still other embodiments, an alkyl group is from 1 to 6 carbon atoms.

“Alkanyl” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent, refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” by itself or as part of another substituent, refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. In some embodiments, the alkyldiyl group is (C₁-C₂₀) alkyldiyl. In other embodiments, the alkyldiyl group is (C₁-C₁₀) alkyldiyl. In still other embodiments, the alkyldiyl group is (C₁-C₆) alkyldiyl. Preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkyleno, defined infra).

“Alkyleno” by itself or as part of another substituent, refers to a straight-chain alkyldiyl group having two terminal monovalent radical centers derived by the removal of one hydrogen atom from each of the two terminal carbon atoms of straight-chain parent alkane, alkene or alkyne. Typical alkyleno groups include, but are not limited to, methano; ethylenos such as ethano, etheno, ethyno; propylenos such as propano, prop[i]eno, propa[1,2]dieno, prop[i]yno, etc.; butylenos such as butano, but[1]eno, but[2]eno, buta[1,3]dieno, but[1]yno, but[2]yno, but[1,3]diyno, etc.; and the like. Where specific levels of saturation are intended, the nomenclature alkano, alkeno and/or alkyno is used. In some embodiments, the alkyleno group is (C₁-C₂₀) alkyleno. In other embodiments, the alkyleno group is (C₁-C₁₀) alkyleno. In still other embodiments, the alkyleno group is (C₁-C₆) alkyleno. Preferred are straight-chain saturated alkano groups, e.g., methano, ethano, propano, butano and the like.

“Acyl” by itself or as part of another substituent, refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein. Representative examples include, but are not limited to formyl, acetyl, cyclohexylcarbonyl, cyclohexylmethylcarbonyl, benzoyl, benzylcarbonyl and the like.

“Acylamino” by itself or as part of another substituent, refers to a radical —NR³¹C(O)R³², where R³¹ and R³² are each independently hydrogen, alkyl, cycloalkyl, cycloheteroalkyl, aryl, arylalkyl, heteroalkyl, heteroaryl, heteroarylalkyl, as defined herein. Representative examples include, but are not limited to, formylamino, acetylamino, cyclohexylcarbonylamino, cyclohexylmethyl-carbonylamino, benzoylamino, benzylcarbonylamino and the like.

“Alkoxy” by itself or as part of another substituent, refers to a radical —OR³³ where R³³ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent, refers to a radical —C(O)OR³⁴ where R³⁴ represents an alkyl or cycloalkyl group as defined herein.

“Aryl” by itself or as part of another substituent, refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. Preferably, an aryl group comprises from 6 to 20 carbon atoms, more preferably from 6 to 12 carbon atoms.

“Arylalkyl” by itself or as part of another substituent, refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl e.g., the alkanyl, alkenyl or alkynyl moiety of the arylalkyl group is (C₁-C₁₀) and the aryl moiety is (C₆-C₂₀). In other embodiments, the arylalkyl group is (C₆-C₂₀) arylalkyl, e.g., the alkanyl, alkenyl or alkynyl moiety of the arylalkyl group is (C₁-C₈) and the aryl moiety is (C₆-C₁₂).

“Cycloalkyl” by itself or as part of another substituent, refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In some embodiments, the cycloalkyl group is (C₃-C₁₀) cycloalkyl. In other embodiments, the cycloalkyl group is (C₃-C₇) cycloalkyl.

“Cycloheteroalkyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl, Heteroalkanyl, Heteroalkyldiyl and Heteroalkyleno” by themselves or as part of another substituent, refer to alkyl, alkanyl, alkenyl, alkynyl, alkyldiyl and alkyleno groups, respectively, in which one or more of the carbon atoms (and any associated hydrogen atoms) are each independently replaced with the same or different heteroatomic groups. Typical heteroatomic groups which can be included in these groups include, but are not limited to, —O—, —S—, —O—O—, —S—S—, —O—S—, —NR³⁵R³⁶—, ═N—N═, —N═N—, —N═N—NR³⁷R³⁸, —PR³⁹—, —P(O)₂—, —POR⁴⁰—, —O—P(O)₂—, —SO—, —SO₂—, —SnR⁴¹R⁴²— and the like, where R³⁵, R³⁶, R³⁷, R³⁸, R³⁹, R⁴⁰, R⁴¹ and R⁴² are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group is from 5-20 membered heteroaryl. In other embodiments, the heteroaryl group is from 5-10 membered heteroaryl. Preferred heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In some embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In other embodiments, a 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Parent Aromatic Ring System” by itself or as part of another substituent, refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System” by itself or as part of another substituent, refers to a parent aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Pharmaceutically acceptable salt” refers to a salt of a compound of the invention, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound is administered.

“Patient” includes humans. The terms “human” and “patient” are used interchangeably herein.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Substituted” refers to a group in which one or more hydrogen atoms are independently replaced with the same or different substituent(s). Typical substituents include, but are not limited to, -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is independently a halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. Preferably, substituents include -M, —R⁶⁰, ═O, —OR⁶⁰, —SR⁶, —S—, ═S, —NR⁶⁰R⁶¹, ═NR⁶⁰, CF₃, —CN, —OCN, —SCN, —NO, —NO₂, ═N₂, —N₃, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹, more preferably, -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻, even more preferably, -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, etc., of the patient to be treated.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### 4.2 Compounds of Structural Formula (I) Useful in Formulations and Solid Dosage Forms

In some embodiments, thiomolybdate and thiotungstate compounds which may be useful in the formulations and solid dosage form of the present invention include compounds of structural formula (I): 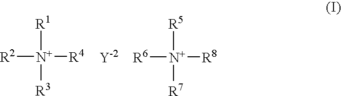
or solvates, hydrates or N-oxides thereof wherein:

R¹, R², R³, R⁵, R⁶ and R⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl;
R⁴ and R⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl or are absent when N is part of an aromatic ring;

optionally, R¹ and R² taken together are alkyldiyl, substituted alkyldiyl, heteroalkyldiyl or substituted heteroalkyldiyl;

optionally, R⁵ and R⁶ taken together are alkyldiyl, substituted alkyldiyl, heteroalkyldiyl or substituted heteroalkyldiyl;

optionally, R¹ and R² taken together, R² and R³ taken together and R² and R⁴ taken together are alkyldiyl, substituted alkyldiyl, heteroalkyldiyl or substituted heteroalkyldiyl;

optionally, R⁵ and R⁶ taken together, R⁶ and R⁷ taken together and R⁶ and R⁸ taken together are alkyldiyl, substituted alkyldiyl, heteroalkyldiyl or substituted heteroalkyldiyl;

optionally, R³ and R⁷ taken together are alkyldiyl, substituted alkyldiyl, heteroalkyldiyl or substituted heteroalkyldiyl; and

Y⁻² is (MoS₄)⁻², (Mo₂S₁₂)⁻², (Mo₂S₉)⁻², (Mo₂S₇)⁻², (Mo₂S₈)⁻², (Mo₂S₁₁)⁻², Mo₂S₆)⁻² or (Mo₂S₁₃)⁻², Y⁻² is (WS₄)⁻², (W₂S₁₂)⁻², (W₂S₉)⁻², (W₂S₇)⁻², (W₂S₈)⁻², W₂S₁₁)⁻², (W₂S₆)⁻² or (W₂S₁₃)⁻²;

In other embodiments, 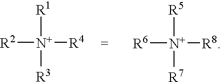
In some of these embodiments, Y is (MoS₄)⁻² or (WS₄)⁻².

In some embodiments, at least one of R¹, R², R³ and R⁴ is not alkyl. In other embodiments, R¹, R² and R⁴ are hydrogen, alkanyl or substituted alkanyl. In some of these embodiments, R¹, R² and R⁴ are hydrogen, methyl or ethyl.

In still other embodiments, R¹ and R² are alkanyl. In some of these embodiments, R¹ and R² are methyl or ethyl.

In still other embodiments, R¹ is alkanyl, substituted alkanyl, alkenyl, substituted alkenyl, aryl, substituted aryl, arylalkyl, substituted arylalkyl, cycloalkyl or substituted cycloalkyl. In some of these embodiments, R¹ and R² taken together are alkyleno, substituted alkyleno, heteroalkyleno or substituted heteroalkyleno. In other of these embodiments, R¹ and R² taken together are alkyleno or heteroalkyleno.

In still other embodiments, R¹ and R² taken together, R² and R³ taken together and R² and R⁴ taken together are alkyleno, substituted alkyleno, heteroalkyleno or substituted heteroalkyleno. In some of these embodiments, R¹ and R² taken together, R² and R³ taken together and R² and R⁴ taken together are alkyleno. In other of these embodiments, R¹(R²)(R³)(R⁴)N has the structure:

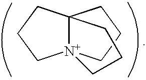

In still other embodiments, R³ and R⁷ taken together are alkyleno, substituted alkyleno, heteroalkyleno or substituted heteroalkyleno. In some of these embodiments, R³ and R⁷ taken together are alkyleno or heteroalkyleno.

In still other embodiments, R¹, R² and R⁴ are hydrogen, alkanyl or substituted alkanyl and R³ is alkyl, substituted alkyl, alkenyl, aryl, arylalkyl, cycloalkyl or R³ and R⁷ taken together are alkyleno, substituted alkyleno, heteroalkyleno or substituted heteroalkyleno. In some of these embodiments, R¹, R² and R⁴ are methyl or ethyl and R³ is alkyl, substituted alkyl, alkenyl, aryl, arylalkyl, cycloalkyl or R³ and R⁷ taken together are alkyleno or heteroalkyleno. In other of these embodiments, R¹, R² and R⁴ are methyl or ethyl and R³ is alkyl, substituted alkyl, alkenyl, aryl, arylalkyl or cycloalkyl.

In still other embodiments, R(R²)(R³)(R⁴)N is

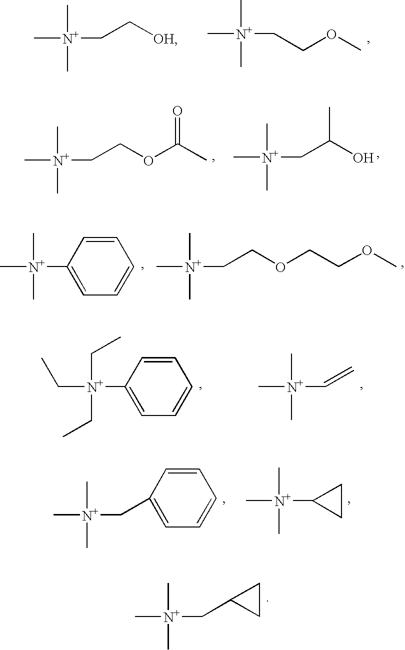

In still other embodiments, R¹(R²)(R³)(R⁴)N is

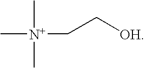

In still other embodiments, R¹(R²)(R³)(R⁴)N is

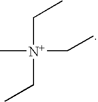

In still other embodiments, R¹, R² and R⁴ are methyl or ethyl and R³ and R⁷ taken together are alkyleno or heteroalkyleno. In some of these embodiments, R¹(R²)(R³)(R⁴)N has the structure:

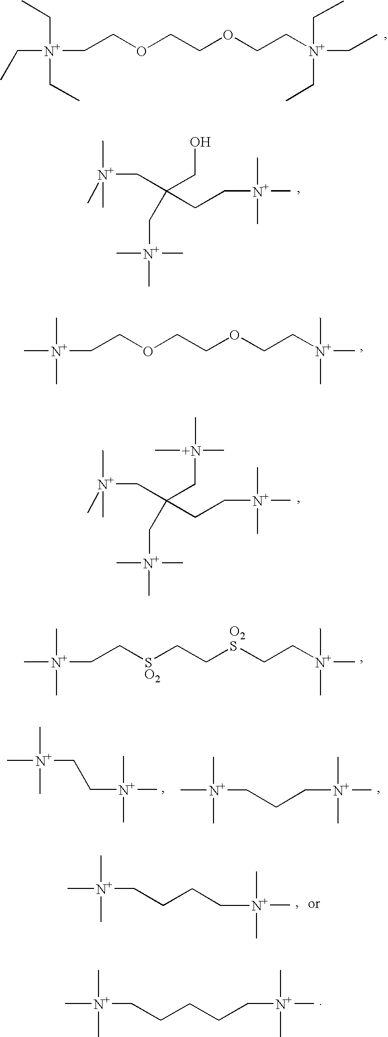

In still other embodiments, R¹, R² and R⁴ are hydrogen and R³ is substituted alkyl, cycloalkyl or substituted heteroaryl or R³ and R⁷ taken together are alkyleno. In still other embodiments, R¹ and R² are alkanyl and R³ and R⁴ are alkyl, substituted alkyl, aryl, arylalkyl or alkyleno. In some of these embodiments, R¹ and R² are methyl or ethyl and R³ and R⁴ are alkyl, substituted alkyl, aryl, arylalkyl or alkyleno.

In still other embodiments, R¹(R²)(R³)(R⁴)N are

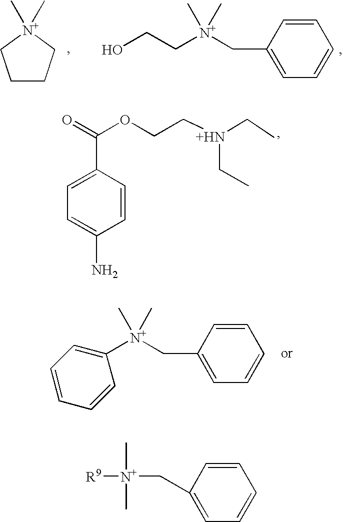

wherein R⁹ is a mixture of straight chain alkanyl groups which have at least eight carbon atoms and not more than eighteen carbon atoms.

In still other embodiments, R¹, R² and R⁴ are hydrogen and R³ is substituted alkyl, substituted heteroaryl, cycloalkyl or alkyleno. In some of these embodiments, R¹(R²)(R³)(R⁴)N has the structure:

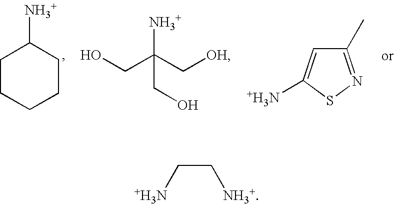

In still other embodiments, R¹ and R² taken together are alkyleno, substituted alkyleno, heteroalkyleno or substituted heteroalkyleno, R³ is alkyl or substituted alkyl and R⁴ is hydrogen or is absent. In some of these embodiments, R¹(R²)(R³)N or R¹(R²)(R³)(R⁴)N has the structure:

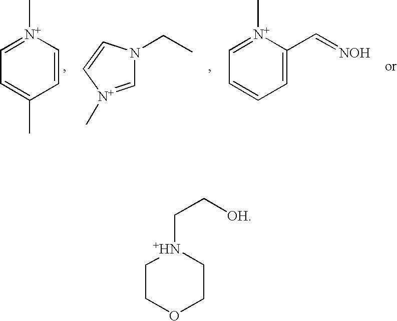

### 4.3 Synthesis

The compounds of Formula (I) may be obtained via conventional synthetic methods illustrated in Schemes 1 and 2. Starting materials useful for preparing compounds of the invention and intermediates thereof are commercially available or can be prepared by well-known synthetic methods. For example, ammonium thiomolybdate may be purchased from well-known chemical suppliers (e.g., Aldrich Chemical Company, Milwaukee, Wis.). Substituted ammonium salts (e.g., ammonium hydroxide and ammonium halides) may be either purchased from commercial sources or may be readily synthesized using well-known synthetic methods (Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995). Other methods for synthesis of the compounds described herein and/or starting materials are either described in the art or will be readily apparent to the skilled artisan. Accordingly, the methods presented in Schemes 1 and herein are illustrative rather than comprehensive.

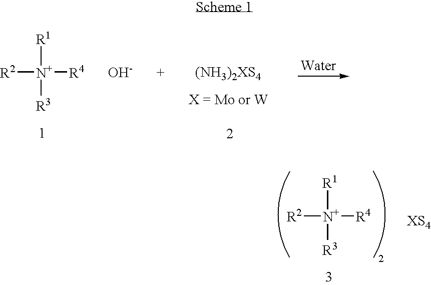

As shown above, in Scheme 1, addition of a quaternary ammonium hydroxide to thiomolybdate in the presence of water leads to cation exchange (equilibrium to product is driven by removal of volatile ammonia) to provide the desired thiomolybdate or thiotungstate derivative.

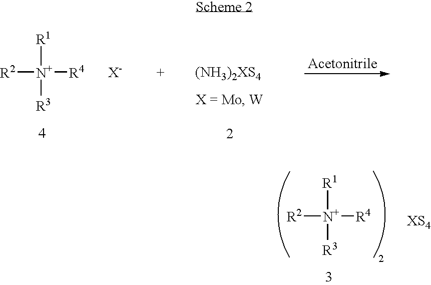

As shown above, in Scheme 2, addition of a quaternary ammonium halide to thiomolybdate in the presence of acetonitrile leads to cation exchange (equilibrium to product is driven by formation of ammonium halide) to provide the desired thiomolybdate or thiotungstate derivative.

Thiomolybdate or thiotungstate derivatives where the ammonium counterions are different may be prepared from compounds 3 through by treating with one equivalent of another ammonium counterion. Although not wishing to be bound by theory such a reaction would be expected to produce a statistical mixture of products.

### 4.4 Formulations and Solid Dosage Forms

Formulations of thiomolybdate or thiotungstate compounds are disclosed herein. The formulations comprise a thiomolybdate or thiotungstate compounds, a pharmaceutically acceptable solvent and a matrix material. Thiomolybdate and thiotungstate compounds which may be used in the dosage forms and formulations have been described in Section 4.2 above. In some embodiments, the formulations are homogeneous solutions or dispersions.

Pharmaceutically acceptable solvents include water, aqueous buffer, organic solvent or mixtures thereof. The pharmaceutically acceptable solvent may be an aqueous buffer, organic solvent or mixture thereof. The pharmaceutically acceptable solvent may also be a mixture of an aqueous buffer and organic solvent.

Representative organic solvents include acetic acid, acetaldehyde, dimethyl acetal, acetone, chloroform, chlorofluorocarbons, dichloromethane, dipropyl ether, ethyl ether, diisopropyl ether, formamide, N,N-dimethyl formamide, dimethyl sulfoxide, dioxane, ethanol, ethyl acetate, ethyl formate, ethyl vinyl ether, methyl ethyl ketone, glycerol, heptane, hexane, isopropanol, methanol, isopropanol, butanol, triethylamine, nitromethane, octane, pentane, tetrahydrofuran, toluene, 1,1,1-trichlorethane, 1,1,2-trichloroethylene, xylene and combinations thereof. In some embodiments, the organic solvent is methanol, ethanol, dimethylformamide or dimethylsulfoxide. In other embodiments, the organic solvent is methanol or ethanol.

In other embodiments, the pharmaceutically acceptable solvent is an aqueous buffer. In some embodiments, the buffer is Tris, sodium phosphate, sodium bicarbonate, sodium borate, arginine, lysine, glycine, ethanolamine or mixtures thereof.

In other embodiments, the aqueous buffer has a pH between about 7.00 and about 14.00. In still other embodiments, the aqueous buffer has a pH between about 7.00 and about 13.0. In still other embodiments, the aqueous buffer has a pH between about 7.00 and about 12.0. In still other embodiments, the aqueous buffer has a pH between about 8.00 and about 12.0. In still other embodiments, the aqueous buffer has a pH between about 9.00 and about 12.0. In still other embodiments, the aqueous buffer has a pH between about 10.00 and about 12.0. In still other embodiments, the aqueous buffer has a pH between about 11.00 and about 12.0.

The aqueous buffer may include a chelating agent. In some embodiments, the chelating agent is a polycarboxylate, such as, for example, ethylenediaminetetraacetic acid, [ethylenebis(oxyethylenenitrilo)]tetraacetic acid, and 1,2-bis(2-aminophenoxy)ethane-N,N,N′,N′-tetraacetic acid or diethylenetriaminepentaacetic acid. In other embodiments, the polycarboxylate is ethylenediaminetetraacetic acid. In still other embodiments, the buffer is about 10.0 mm Tris at about pH 11.0 and about 0.1% EDTA.

In still other embodiments, the concentration of thiomolybdate or thiotungstate compound is between about 1.0 μg/ml and about 350.0 μg/ml. In still other embodiments, the concentration of thiomolybdate or thiotungstate compound is between about 5.0 μg/ml and about 125.0 μg/ml. In still other embodiments, the concentration of thiomolybdate or thiotungstate compound is between about 10.0 μg/ml and about 20.0 μg/ml.

In some embodiments, the matrix material is a water soluble polymer such as, for example, cellulose derivatives (e.g., hydroxypropylmethylcellulose phthalate, hydroxypropylmethylcellulose acetate succinate, carboxymethylcellulose, cellulose acetate, ethylcellulose, methylcellulose, hydroxypropylmethylcellulose, carboxymethylcellulose sodium), methacrylic copolymer, aminoalkylmethacrylate copolymer, poly (vinyl acetal) diethylaminoacetate, polyvinylpyrrolidone, polyvinylpyrrolidone copolymers polyvinyl alcohol, dextrin, pullulan, sodium alginate, alginate, gelatin, starch, lecithin, glucomannan, polyalkylene glycols, polyvinyl alcohols, polysaccharides, polyvinyl acetates, polyacrylic acids, or proteoglycans. In other embodiments, the water soluble polymer is a polyalkylene glycol, polyvinyl alcohol, polysaccharide, polyvinyl acetate, polyacrylic acid, or proteoglycan. In still other embodiments, the water soluble polymer is a polyalkylene glycol, even more preferably, a polyethylene glycol.

In some embodiments, the pharmaceutically acceptable solvent is an aqueous buffer containing ethylenediaminetetraacetic acid and the matrix material is PEG 4000. In other embodiments, the buffer is Tris, lysine, arginine, glycine or triethanolamine. In still other embodiments, the pH is between about 10.00 and about 12.00. In still other embodiments, the pH is between about 11.00 and about 11.50. In still other embodiments, the buffer is 10 mm Tris at pH 11.00. In still other embodiments, the buffer is 50 mm glycine, pH 11.50.

The following compositions may be particularly useful. In some embodiments, the thiomolybdate or thiotungstate compound is between about 3% and about 10%, EDTA is between about 0.01% and about 0.10%, Tris.HCl is between about 0.01% and about 0.10%, deionized water is between about 30% and about 50% and PEG 4000 is between about 45% and about 65% on a weight by weight basis. In other embodiments, the thiomolybdate or thiotungstate compound is between about 4% and about 8%, EDTA is between about 0.02% and about 0.08%, Tris.HCl is between about 0.01% and about 0.10%, deionized water is between about 35% and about 45% and PEG 4000 is between about 50% and about 60% on a weight by weight basis. In still other embodiments, the thiomolybdate or thiotungstate compound is about 6.1%, EDTA is about 0.03%, Tris.HCl is about 0.05%, deionized water is about 38.38% and PEG-4000 is about 55.34% on a weight by weight basis.

In still other embodiments, the thiomolybdate or thiotungstate compound is between about 3% and about 10%, EDTA is between about 0.01% and about 0.10%, deionized water is between about 30% and about 50%, PEG 4000 is between about 45% and about 65% on a weight by weight basis and the glycine concentration is between about 25 mm and about 75 mm. In still other embodiments, the thiomolybdate or thiotungstate compound is between about 4% and about 8%, EDTA is between about 0.02% and about 0.08%, deionized water is between about 35% and about 40% and PEG 4000 is between about 50% and about 60% on a weight by weight basis and the glycine concentration is between about 40 mm and about 60 mm. In still other embodiments, the thiomolybdate or thiotungstate compound is about 6.1%, EDTA is about 0.03%, deionized water about 38.38% and PEG-4000 is about 55.34% on a weight by weight basis and the glycine concentration is about 50.0 mm. In some of the above embodiments, the thiomolybdate compound is choline thiomolybdate, choline thiotungstate, ammonium thiotungstate or ammonium thiomolybdate.

Conventional additives well known in the art may be used in the formulations disclosed herein. Such additives include, but are not limited to, anti-adherents (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate), anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4-hydroxymethyl-2,6-di-tert-butyl phenol, tocopherol, etc.), binders i.e., agents that impart cohesive properties to powdered materials through particle-particle bonding, (e.g., matrix binders (dry starch, dry sugars), film binders (e.g., polyvinyl pyrrolidine, starch paste, celluloses, bentonite, sucrose), and chemical binders (polymeric cellulose derivatives, (e.g., carboxy methyl cellulose, HPC and HPMC), sugar syrups, corn syrup, water soluble polysaccharides (e.g., acacia, tragacanth, guar and alginates), gelatin, gelatin hydrolysate, agar, sucrose, dextrose and non-cellulosic binders, (e.g., PVP, vinyl pyrrolidone copolymers, pregelatinized starch, sorbitol, and glucose)), coagulants (e.g., alginates, colorants or opaquants, such as titanium dioxide, food dyes, lakes, natural vegetable colorants, iron oxides, silicates, sulfates, magnesium hydroxide and aluminum hydroxide), coolants, such as halogenated hydrocarbons (e.g., trichloroethane, trichloroethylene, dichloromethane, fluorotrichloromethane, etc.), diethylether and liquid nitrogen, cryoprotectants, (e.g., trehelose, phosphates, citric acid, tartaric acid, gelatin, dextran, mannitol, etc.), diluents or fillers (e.g., lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate), dextrose disintegrants or super disintegrants, (e.g., croscarmellose sodium, starch, starch derivatives, clays, gums, cellulose, cellulose derivatives, alginates, crosslinked polyvinylpyrrolidone, sodium starch glycolate, microcrystalline cellulose, etc.), hydrogen bonding agents, such as magnesium oxide, flavorants or desensitizers (e.g., spray-dried flavors, essential oils, ethyl vanillin, etc., ion-exchange resins (e.g., styrene/divinyl benzene copolymers, quaternary ammonium compounds, etc.), plasticizers, (e.g., citrate esters (e.g., triethyl citrate, acetyl triethyl citrate, acetyltributyl citrate), acetylated monoglycerides, glycerin, triacetin, phthalate esters (e.g., diethyl phthalate, dibutyl phthalate), castor oil, sorbitol, dibutyl seccate, etc.) preservatives (e.g., ascorbic acid, boric acid, sorbic acid, benzoic acid and salts thereof, parabens, phenols, benzyl alcohol, quaternary ammonium compounds, etc.) solvents (e.g., alcohols, ketones, esters, chlorinated hydrocarbons, water), sweeteners (e.g., maltose, sucrose, glucose, sorbitol, glycerin and dextrins, aspartame, saccharine, saccharine salts, thickeners (viscosity modifiers, thickening agents), such as sugars, polyvinylpyrrolidone, cellulosics, polymers and alginates, proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, carnuaba wax, fatty acids (e.g., stearic acid, hydroxystearic acid), fatty alcohols, sugars, shellacs, such as those based on sugars (e.g., lactose, sucrose, dextrose) or starches, polysaccharide-based shellacs (e.g., maltodextrin and maltodextrin derivatives, dextrates, cyclodextrin and cyclodextrin derivatives), cellulosic-based shellacs (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate, trimellitate, carboxymethylethyl cellulose, hydroxypropylmethyl cellulose phthalate), inorganics, such as dicalcium phosphate, hydroxyapitite, tricalcium phosphate, talc and titania, polyols, such as mannitol, xylitol and sorbitol; polyethylene glycol esters and polymers, such as alginates, poly(lactide coglycolide), gelatin, crosslinked gelatin, and agar-agar.

It should be appreciated that considerable overlap exists between the above-listed additives in common usage, since a given additive is often classified differently by different practitioners in the field, or is commonly used for any of several different functions. Thus, the above-listed additives should be taken as merely exemplary, and not limiting, of the types of additives that can be included in the formulations of the present invention. The amounts of such additives can be readily determined by one skilled in the art, according to the particular properties desired.

### 4.5 Solid Dosage Forms and Dispersions and Preparation Thereof

Also disclosed herein are solid dispersions and solid dosage forms, both of which comprise a thiomolybdate or thiotungstate compound and a matrix material. Solid dispersions or solid dosage forms are preferably formed in a container (e.g., a capsule) by addition of a solution and/or dispersion comprising a thiomolybdate or thiotungstate compound, a matrix material and a pharmaceutically acceptable solvent to the container followed by removal of the pharmaceutically acceptable solvent. Exemplary thiomolybdate or thiotungstate compounds, matrix materials and pharmaceutical compositions and combinations thereof have been described above.

Importantly, the container should be stable to the solution and/or dispersion of thiomolybdate or thiotungstate compound, matrix material and pharmaceutically acceptable solvent. In some embodiments, the container is a capsule which, ideally, is stable to the solution and/or dispersion of thiomolybdate or thiotungstate compound, matrix material and pharmaceutically acceptable solvent. Evaluating the stability of a capsule to the solution and/or dispersion of thiomolybdate or thiotungstate compound, matrix material and pharmaceutically acceptable solvent is well within the ambit of those of skill in the art.

A large variety of capsules are known to the skilled artisan including, for example, hard and soft containers or shells comprised of gelatin (The Science and Practice of Pharmacy, Remington, Nineteenth Edition, pp. 1642-1646, 1995) cellulose Cade et al., United States Patent Application Publication No. 2002/018790), kappa and iota carregan (Fonkwe et al., United States Patent Application Publication No. 2003/0138482), pH dependent polymers (Chen et al., United States Patent Application Publication No. 2003/0161872), hard capsules comprised of polymers of vinyl esters and polyethers (Angel et al., United States Patent Application Publication No. 2001/0036471), soft capsules made of iota-carrageenan (Tanner et al., United States Patent Application Publication No. 2002/0081331).

In some embodiments, the capsule is comprised of a alkyl cellulose, more preferably, a hydroxydialkyl cellulose, even more preferably, a hydroxypropylalkyl cellulose and most preferably, hydroxypropyl methyl cellulose. Exemplary thiomolybdate or thiotungstate compounds, matrix materials and pharmaceutical compositions and combinations thereof have been described above.

In some embodiments, the solid dosage form is made by adding a solution and/or dispersion comprising a thiomolybdate or thiotungstate compound, a matrix material and a pharmaceutically acceptable solvent to a container and evaporating the solvent. In some embodiments, the container is a capsule. The solvent may be evaporated by any method known to those of skill in the art but is preferably, evaporated under reduced pressure.

### 4.6 Therapeutic Uses

A solid dosage form of a compound of structural formula (I) may be administered to a patient, preferably, a human, suffering from a disease characterized by aberrant vascularization, copper metabolism disorders, neurodegenerative disorders and obesity. Aberrant vascularization includes abnormal neovascularization such as the formation of new blood vessels, larger blood vessels, more branched blood vessels and any other mechanism, which leads to an increased blood carrying capacity to a diseased tissue or site. The solid dosage forms may be used to treat aberrant vascularization.

Preferably, diseases characterized by aberrant vascularization include cancer (e.g., any vascularized tumor, preferably, a solid tumor, including but not limited to, carcinomas of the lung, breast, ovary, stomach, pancreas, larynx, esophagus, testes, liver, parotid, bilary tract, colon, rectum, cervix, uterus, endometrium, kidney, bladder, prostrate, thyroid, squamous cell carcinomas, adenocarcinomas, small cell carcinomas, melanomas, gliomas, neuroblastomas, sarcomas (e.g., angiosarcomas, chondrosarcomas)), arthritis, diabetes, arteriosclerosis, arteriovenous, malformations, corneal graft neovascularization, delayed wound healing, diabetic retinopathy, age related macular degeneration, granulations, burns, hemophilic joints, rheumatoid arthritis, hypertrophic scars, neovascular glaucoma, nonunion fractures, Osier Weber Syndrome, psoriasis, granuloma, retrolental fibroplasia, pterygium, scleroderma, trachoma, vascular adhesions, ocular neovascularization, parasitic diseases, hypertrophy following surgery, inhibition of hair growth, macular degeneration (including both wet and dry type), rheumatoid arthritis and osteoarthritis. In some embodiments, diseases characterized by aberrant vascularization include cancer, macular degeneration and rheumatoid arthritis.

Further, a solid dosage form of a compound of structural formula (I) may be administered to a patient, preferably, a human, suffering from a disease associated with copper metabolism disorders (e.g., Wilson's disease) to treat such a disease.

Still further, a solid dosage form of a compound of structural formula (I) may be administered to a patient, preferably, a human, to treat obesity. The compounds of structural formula (1) may be also used to reduce levels of inflammatory cytokines (e.g., TNF-α, TNF-β, IL-8, etc.) and plasminogen activator inhibitor, which may be associated with angiogenesis and obesity (Loskutoff et al., Ann. N.Y. Acad. Sci., 2000, 902:272-281; Pan et al., Cancer Res., 2002, 62:4854-4859; Hanada et al., Cytokine Growth Factor Rev. 2002, 13: 413-421; Chen et al., Science 2002, 296:1634-5; Miyake et al., J. Neuropathol. Exp. Neurol. 59:18-28; Koch et al., Science 1992, 258:1798-801; Osawa et al., Infect. Immun. 2002, 70:6294-6301; Bajou et al., Nat. Med. 1998, 4 923-8).

Still further, a solid dosage form of a compound of structural formula (I) may be administered to a patient, preferably a human, suffering from a neurodegenerative disorder, to treat a neurodegenerative disorder. Elevated levels of copper have been reported in the art to mediate the pathobiology of various neurodegenerative disorders including Alzheimer's disease, amyotrophic lateral sclerosis (ALS) and prion disease (Llanos et al., DNA Cell Biol. 2002, 21: 259-270; Carri et al., Funct. Neurol. 2001, 16:181-188; Perry et al., CNS Drugs 2002, 16:339-352; Kowalik-Jankowska et al., Environ Health Perspect. 2002, 5: 869-870; Maynard et al., J. Biol. Chem. 2002, Sep. 4; Gnjec et al., Front Biosci. 2002, 16-23; Strausak et al., Brain Res. Bull. 2001 55: 175-185; Brown, Brain Res. Bull. 2001, 55:165-173; Brown, Biochem. Soc. Trans. 2002, 30:742-745).

Further, in certain embodiments, a solid dosage form of a compound of structural formula (I) are administered to a patient, preferably a human, as a preventative measure against various diseases or disorders characterized by aberrant vascularization, copper metabolism disorders, neurodegenerative disorders and obesity. Accordingly, a solid dosage form of a compound of structural formula (I) may be used for the prevention of one disease or disorder and concurrently treating another (e.g., preventing Wilson's disease or Alzheimer's disease while treating cancer).

### 4.7 Therapeutic/Prophylactic Administration

The solid dosage form of a compound of structural formula (I) may be advantageously used in human medicine. As previously described in Section 4.6 above, the solid dosage form of a compound of structural formula (I) is useful for the treatment or prevention of various diseases characterized by aberrant vascularization, copper metabolism disorders, neurodegenerative disorders and obesity.

When used to treat or prevent the above diseases or disorders, a solid dosage form of a compound of structural formula (I) may be administered or applied singly, or in combination with other agents. The solid dosage form of a compound of structural formula (I) may also be administered or applied singly, in combination with other pharmaceutically active agents (e.g., other anti-cancer agents, other anti-angiogenic agents, other chelators such as zinc, penicillamine, etc. and other anti-obesity agents), including other solid dosage forms and/or formulations of a compound of structural formula (I).

Methods of treatment and prophylaxis by administration to a patient of a therapeutically effective amount of a solid dosage form of a compound of structural formula (I) are provided herein. The patient may be an animal, more preferably, is a mammal and most preferably is a human.

In some embodiments, the solid dosage form of a compound of structural formula (I), is preferably administered orally which results in the release of a compound of structural formula (I) into the bloodstream. In other embodiments, The solid dosage forms of the invention are delivered via sustained release systems, including oral sustained release systems. In some embodiments, a pump may be used (See, Langer, supra; Sefton, 1987, CRC Crit Ref Biomed Eng. 14:201; Saudek et al., 1989, N. Engl. J. Med. 321:574).

In other embodiments, polymeric materials can be used (see “Medical Applications of Controlled Release,” Langer and Wise (eds.), CRC Pres., Boca Raton, Fla. (1974); “Controlled Drug Bioavailability,” Drug Product Design and Performance, Smolen and Ball (eds.), Wiley, New York (1984); Langer et al., 1983, J Macromol. Sci. Rev. Macromol. Chem. 23:61; see also Levy et al., 1985, Science 228: 190; During et al., 1989, Ann. Neurol. 25:351; Howard et al., 1989, J. Neurosurg. 71:105). In still other embodiments, polymeric materials are used for oral sustained release delivery. Exemplary polymers include sodium carboxymethylcellulose, hydroxypropylcellulose, hydroxypropylmethylcellulose and hydroxyethylcellulose. Other exemplary cellulose ethers have been described (Alderman, Int. J. Pharm. Tech. & Prod. Mfr., 1984, 5(3) 1-9). Factors affecting drug release are well known to the skilled artisan and have been described in the art (Bamba et al., Int. J. Pharm., 1979, 2, 307).

In other embodiments, enteric-coated preparations can be used for oral sustained release administration. Exemplary coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In still other embodiments, osmotic delivery systems are used for oral sustained release administration (Verma et al., Drug Dev. Ind. Pharm., 2000, 26:695-708). In other embodiments, OROS™ osmotic devices are used for oral sustained release delivery devices (Theeuwes et al., U.S. Pat. No. 3,845,770; Theeuwes et al., U.S. Pat. No. 3,916,899).

### 4.8 Therapeutic Doses

A solid dosage form will generally be used in an amount effective to achieve the intended purpose. For use to treat or prevent diseases or disorders characterized by aberrant vascularization, copper metabolism disorders, neurodegenerative disorders and obesity the compounds of structural Formula (I) and/or pharmaceutical compositions thereof, are administered or applied in a therapeutically effective amount.

The amount of a solid dosage form that will be effective in the treatment of a particular disorder or condition disclosed herein will depend on the nature of the disorder or condition, and can be determined by standard clinical techniques known in the art as previously described. In addition, in vitro or in vivo assays may optionally be employed to help identify optimal dosage ranges. The amount of a solid dosage form administered will, of course, be dependent on, among other factors, the subject being treated, the weight of the subject, the severity of the affliction, the manner of administration and the judgment of the prescribing physician.

For example, the dosage may be delivered in a solid dosage form by a single administration, by multiple applications or controlled release. In some embodiments, the solid dosage are delivered by oral sustained release administration. In these embodiments, the compounds are administered twice per day. In other of these embodiments, the compounds are administered once per day. Dosing may be repeated intermittently, may be provided alone or in combination with other drugs and may continue as long as required for effective treatment of the disease state or disorder.

Suitable dosage ranges for oral administration depend on the potency of the drug, but are generally between about 0.001 mg to about 200 mg of a compound per kilogram body weight. Dosage ranges may be readily determined by methods known to the artisan of ordinary skill.

### 4.9 Combination Therapy

In certain embodiments, the solid dosage form described herein can be used in combination therapy with at least one other therapeutic agent. The solid dosage form and the therapeutic agent can act additively or synergistically. In some embodiments, a solid dosage form of the invention is administered concurrently with the administration of another therapeutic agent. In other embodiments, a solid dosage form is administered prior or subsequent to administration of another therapeutic agent.

In particular, in some embodiments, the solid dosage forms can be used in combination therapy with other chemotherapeutic agents (e.g., alkylating agents (e.g., nitrogen mustards (e.g., cyclophosphamide, ifosfamide, mechlorethamine, melphalen, chlorambucil, hexamethylmelamine, thiotepa), alkyl sulfonates (e.g., busulfan, nitrosoureas, triazines) antimetabolites (e.g., folic acid analogs, pyrimidine analogs (e.g., fluorouracil, floxuridine, cytosine arabinoside, etc.), purine analogs (e.g., mercaptopurine, thiogunaine, pentostatin, etc.), natural products (e.g., vinblastine, vincristine, etoposide, tertiposide, dactinomycin, daunorubicin, doxurubicin, bleomycin, mithrmycin, mitomycin C, L-asparaginase, interferon alpha), platinum coordination complexes (e.g., cis-platinum, carboplatin, etc.), mitoxantrone, hydroxyurea, procarbazine, hormones and antagonists (e.g., prednisone, hydroxyprogesterone caproate, medroxyprogesterone acetate, megestrol acetate, diethylstilbestrol, ethinyl estradiol, tamoxifen, testosterone propionate, fluoxymesterone, flutamide, leuprolide, etc.), anti-angiogenesis agents or inhibitors (e.g., angiostatin, retinoic acids and paclitaxel, estradiol derivatives, thiazolopyrimidine derivatives, etc.), apoptosis-inducing agents (e.g., antisense nucleotides that block oncogenes which inhibit apoptosis, tumor suppressors, TRAIL, TRAIL polypeptide, Fas-associated factor 1, interleukin-1β-converting enzyme, phosphotyrosine inhibitors, RXR retinoid receptor agonists, carbostyril derivatives, etc.), chelators (penicillamine, zinc, trientine, etc.) and other anti-obesity agents.

### 5. EXAMPLES

The invention is further defined by reference to the following examples, which describe in detail, preparation of formulations and solid dosage forms. It will be apparent to those skilled in the art that many modifications, both to materials and methods, may be practiced without departing from the scope of the invention.

### Example 1

### Preparation of Capsules of Choline Thiomolybdate

Glycine buffer was prepared by dissolving 3.7546 g glycine free base, 1.005 g disodium EDTA dihydrate in 900 mL of distilled, adjusting the pH to 11.5 by 2N NaOH and bringing the volume to 1000 mL. The buffer was sparged with argon for 15 minutes and sealed with argon in the headspace in a glass bottle for storage. Choline thiomolybdate (54.3 g) was dissolved in glycine buffer (356.7 g) made as described above, by mixing with a homogenizer at 200-500 RPM with an argon blanket over the liquid surface. PEG 4000 (488.9 g) was then added and dissolved by mixing at 3000-6000 RPM with the homogenizer, with an argon blanket over the liquid surface to form a uniform paste. Hydroxypropyl methyl cellulose capsules were filled by pipette, capped and dried under vacuum (50 mTorr) at 30° C. for 72 hours to provide solid capsule dosage forms of choline thiomolybdate after the capped capsules were cooled at about 0-5° C. for about 1 hour to provide a solid encapsulated solid dosage form of choline thiomolybdate.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims. All publications and patents cited herein are incorporated by reference.

What is claimed is:

1. A formulation comprising a thiomolybdate or thiotungstate compound, a pharmaceutically acceptable solvent and a matrix material.

2. The formulation of claim 1, wherein the thiomolybdate or thiotungstate compound is a compound of structural Formula (I): 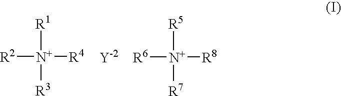
or a solvate or hydrate thereof wherein:
R¹, R², R³, R⁵, R⁶ and R⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl;
R⁴ and R⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, heteroalkyl or substituted heteroalkyl or are absent when N is part of an aromatic ring;
optionally, R¹ and R² taken together are alkyldiyl, substituted alkyldiyl, heteroalkyldiyl or substituted heteroalkyldiyl;
optionally, R⁵ and R⁶ taken together are alkyldiyl, substituted alkyldiyl, heteroalkyldiyl or substituted heteroalkyldiyl;
optionally, R¹ and R² taken together, R² and R³ taken together and R² and R⁴ taken together are alkyldiyl, substituted alkyldiyl, heteroalkyldiyl or substituted heteroalkyldiyl;
optionally, R⁵ and R⁶ taken together, R⁶ and R⁷ taken together and R⁶ and R⁸ taken together are alkyldiyl, substituted alkyldiyl, heteroalkyldiyl or substituted heteroalkyldiyl;
optionally, R³ and R⁷ taken together are alkyldiyl, substituted alkyldiyl, heteroalkyldiyl or substituted heteroalkyldiyl; and
Y⁻² is (WS₄)⁻², (MoS₄)⁻², (Mo₂S₁₂)⁻², (Mo₂S₉)⁻², (Mo₂S₇)⁻², (Mo₂S₈)⁻², (Mo₂S₁₁)⁻², (Mo₂S₆)⁻² or (Mo₂S₁₃)⁻².

3. The formulation of claim 2, wherein is Y⁻² is (WS₄)⁻², (MoS₄)⁻².

4. The formulation of claim 1, wherein the thiomolybdate compound or thiotungstate compound is ammonium thiomolybdate, choline thiomolybdate, ammonium thiotungstate or choline thiotungstate.

5. The formulation of claim 1, wherein the thiomolybdate compound is choline thiomolybdate.

6. The formulation of claim 1, wherein the thiomolybdate compound is ammonium thiomolybdate.

7. The formulation of claim 1, wherein the pharmaceutically acceptable solvent is an aqueous buffer.

8. The formulation of claim 7, wherein the aqueous buffer includes a chelating agent.

9. The formulation of claim 8, wherein the chelating agent is a polycarboxylate.

10. The formulation of claim 9, wherein the polycarboxylate is ethylenediaminetetraacetic acid, [ethylenebis(oxyethylenenitrilo)]tetraacetic acid, and 1,2-bis(2-aminophenoxy)ethane-N,N,N′,N′-tetraacetic acid or diethylenetriaminepentaacetic acid.

11. The formulation of claim 10, wherein the polycarboxylate is ethylenediaminetetraacetic acid.

12. The formulation of claim 1, wherein the pharmaceutically acceptable solvent is an aqueous buffer containing ethylenediaminetetraacetic acid and the matrix material is PEG 4000.

13. The formulation of claim 37, wherein the buffer is Tris, lysine, arginine, glycine or triethanolamine.

14. A formulation comprising a dispersion of a thiomolybdate or thiotungstate compound, a pharmaceutically acceptable solvent and a matrix material.

15. A solid dosage form comprising a thiomolybdate or thiotungstate compound and a matrix material.

16. A capsule dosage form comprising the solid dosage form of claim 15 in a capsule.

17. The capsule dosage form of claim 16, wherein the capsule comprises a hydroxydialkyl cellulose.

18. The capsule dosage form of claim 17, wherein the hydroxydialkyl cellulose is hydroxypropyl methylcellulose.

19. A solid dosage form consisting essentially of a thiomolybdate or thiotungstate compound and a matrix material.

20. A solid dosage form comprising a thiomolybdate or thiotungstate compound, a pharmaceutically acceptable solvent and a matrix material.
